(19) United States
(12) Patent Application Publication
(10) Pub. No.: US 2025/0176816 A1
(43) Pub. Date: Jun. 5, 2025

(54) INTRAORAL IMAGING AID

(71) Applicant: SQRIE CORP., Sakyouku, Kyotoshi, Kyoto (JP)

(72) Inventor: Takahiro OKAMOTO, Sakyouku, Kyotoshi, Kyoto (JP)

(21) Appl. No.: 18/838,283

(22) PCT Filed: Feb. 24, 2022
(86) PCT No.: PCT/JP2022/007546

§ 371 Date: Jan. 16, 2025

(51) Int. Cl. A61B 1/247; A61B 1/00; A61B 1/04

(52) U.S. Cl. CPC A61B 1/247; A61B 1/00128; A61B 1/04

(57) ABSTRACT

The present disclosure provides an oral imaging aid that allows a patient or her or his family to image the inside of the mouth easily, smoothly, and accurately by using a mobile terminal such as a smartphone or tablet. The oral imaging aid includes an insertion plate 2 that is inserted into the mouth, the insertion plate 2 having a reflective surface 21 reflecting and displaying the inside of the mouth; a holder 3 that detachably holds a mobile terminal 100 having an imaging means 101; and a detachable attachment mechanism 4 that detachably attaches the insertion plate 2 to the holder 3 at a predetermined angle.

### CROSS-REFERENCE TO RELATED APPLICATION

This application is a national stage application of International Patent Application No. PCT/JP2022/007546, filed on Feb. 24, 2022, the disclosure of which is incorporated by reference herein in its entirety as part of the present application.

### TECHNICAL FIELD

The present disclosure relates to an intraoral imaging aid that assists with imaging the inside of the mouth during oral inspection.

### BACKGROUND

Conventionally, technology for a device and an aid to assist with imaging the inside of the mouth has been developed. Recently, there has been online medical examination where a patient, etc., images the inside of her or his mouth at home and sends the image such as the still or moving image to a remote hospital, doctor, etc., for analysis of the oral image, allowing for remote oral examination.

In the case of such remote online examination, it is necessary for a patient her or himself or the patient's family to image the patient's mouth. For example, there is growing interest in technology related to an oral imaging aid that enables a patient to use a widely available smartphone to image the inside of her or his mouth (refer to Patent Documents 1 and 2).

As the result, the present disclosure provides an oral imaging aid that enables a patient her or himself or the patient's family to accurately image the inside of the mouth by easily and smoothly using a mobile terminal such as a smartphone or tablet.

The oral aid tool of Patent Document 1 includes an insertion plate to be inserted into the mouth, which at its base end side is formed at an angle greater than 180 degrees but less than 270degrees relative to the surface of the insertion plate: a smartphone holding plate: and a means for fixing a smartphone to the smartphone holding plate.

Furthermore, Patent Document 2 proposes an oral imaging aid that includes a cylindrical main body with a first end having an opening into which an imaging device can be inserted: a second end located opposite the first end with a window that provides a field of view from the inside to the outside of the main body: and an inner circumferential surface that extends from the first end towards the second end and allows the imaging device to slide.

### DOCUMENT IN THE EXISTING ART

### Patent Document

Patent Document 1: JP6806400 B

Patent Document 2: JP2021-040666 A

### SUMMARY

The technical problem solved by the disclosure

To appropriately conduct remote examination of the oral cavity, it is necessary to accurately image the correct location in the mouth. Moreover, to allow a patient her or himself to take the image, there is a demand for a device that is simple to operate.

However, with conventional oral imaging aids like the mentioned above, there has been a problem in that it is often difficult to accurately and cleanly image the inside of the mouth because it is not possible to attach the camera of a mobile terminal such as a smartphone or tablet at the appropriate angle or position relative to the insertion plate,. etc.

The present disclosure has been made in view of the above-mentioned problems and aims to provide an oral imaging aid that allows a patient or her or his family to accurately image the inside of the mouth by easily and smoothly using a mobile terminal such as a smartphone or tablet.

### Solution for Solving the Technical Problem

To solve the above problems, the present disclosure is configured by an oral imaging aid 1 that includes an insertion plate 2 that is inserted into the mouth, the insertion plate 2 having a reflective surface 21 reflecting and displaying the inside of the mouth; a holder 3 that detachably holds a mobile terminal 100 having an imaging means 101; and a detachable attachment mechanism 4 that detachably attaches the insertion plate 2 to the holder 3 at a predetermined angle.

The detachable attachment mechanism 4 may have a clip 41 that is mounted on the base end part of the insertion plate 2; and a detachable attachment guide 42 that is provided on the holder 3 to detachably engage with the clip 41.

Furthermore, the holder 3 may be provided to allow the mounting position of the mobile terminal 100 to be changed.

A handle part 32 may be formed on either or both of the left and the right sides of the holder 3.

The reflective surface 21 of the insertion plate 2 may be treated with an anti-fogging process.

Additionally, the insertion plate 2 may be formed with an alignment marker 5 for alignment in the mouth.

The insertion plate 2 may also be equipped with a light source 6 for illuminating the inside of the mouth.

### Technical Effect

According to the present disclosure including an insertion plate that is inserted into the mouth, the insertion plate having a reflective surface reflecting and displaying the inside of the mouth: a holder that detachably holds a mobile terminal having an imaging means: and a detachable attachment mechanism that detachably attaches the insertion plate to the holder at a predetermined angle, the camera application of a mobile terminal is allowed to be used to image the inside of the mouth by attaching the insertion plate, which is inserted into the mouth, to the holder that holds the mobile terminal at an appropriate angle. As the result, the present disclosure provides an oral imaging aid that enables a patient her or himself or the patient's family to accurately image the inside of the mouth by easily and smoothly using a mobile terminal such as a smartphone or tablet. Additionally, the oral imaging aid can be realized with a simple structure and manufactured at a low cost.

### BRIEF DESCRIPTION OF THE DRAWINGS

FIG. 1A is a photograph of an oral imaging aid according to a first embodiment of the present disclosure viewed from above at an angle to the rear side.

FIG. 1B is a photograph of the oral imaging aid according to the first embodiment of the present disclosure viewed from above at an angle to the front side.

FIG. 2 is a perspective view of the oral imaging aid of FIG. 1A viewed from the rear side.

FIG. 3 is a perspective view of the oral imaging aid of FIG. 1A viewed from the front side.

FIG. 4 is a side view of the oral imaging aid of FIG. 1A.

FIG. 5 is a front view of the oral imaging aid of FIG. 1A.

FIG. 6 is a side view of the oral imaging aid of FIG. 1A.

FIG. 7 is a rear view of the oral imaging aid of FIG. 1A.

FIG. 8 is an explanatory diagram showing the disassembly of the oral imaging aid.

FIG. 9 is an enlarged cross-sectional view along the line A-A of FIG. 5.

FIG. 10 is an explanatory diagram showing the state of use of the oral imaging aid.

FIG. 11 is a flowchart showing the steps for using the oral imaging aid of FIG. 1A.

FIG. 12A is a perspective view of another example of the holder for the oral imaging aid.

FIG. 12B is a front view of the other example of the holder for the oral imaging aid.

FIG. 12C is a side view of the other example of the holder for the oral imaging aid.

FIG. 13 is an explanatory diagram of another example of the insertion plate for the oral imaging aid.

### DESCRIPTION OF REFERENCE NUMERALS

1: Oral imaging aid, 2: Insertion plate, 21: Reflective surface, 3: Holder, 32: Handle part, 4: Detachable attachment mechanism, 41: Clip, 42: Detachable attachment guide, 5: Marker, 6: Light source, 100: Mobile terminal

### DETAILED DESCRIPTION OF THE EMBODIMENTS

Hereinafter, the present disclosure will be described more specifically using preferred embodiments. However, the embodiments described below are merely example embodiments of the disclosure, and the scope of the present disclosure is not limited to these examples.

The oral imaging aid according to the present disclosure is an auxiliary device used when a patient her or himself or the patient's family image the inside of the mouth by using a mobile terminal (portable information terminal) equipped with a camera, such as a smartphone or tablet.

Examples of the mobile terminal 100 used in this embodiment may include a smartphone, camera-equipped mobile phone, tablet, or any other portable communication terminal with a camera.

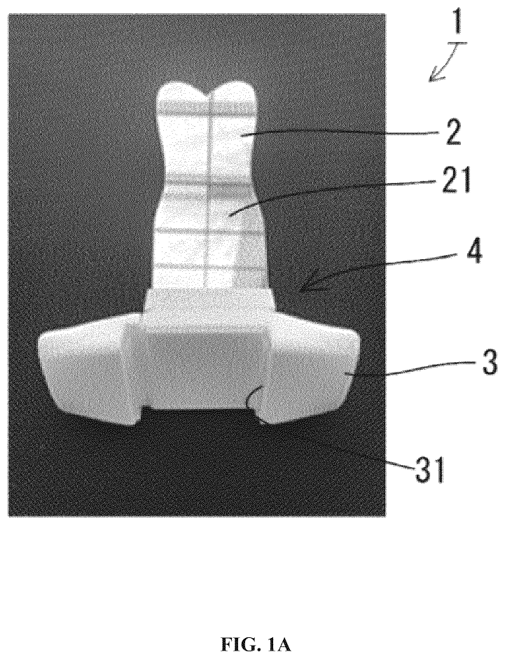

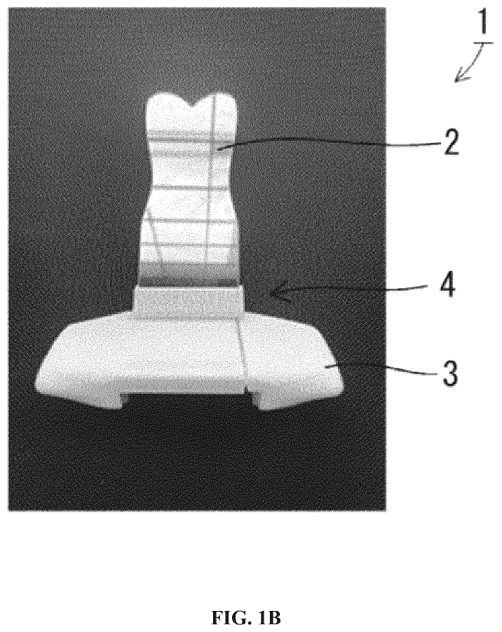

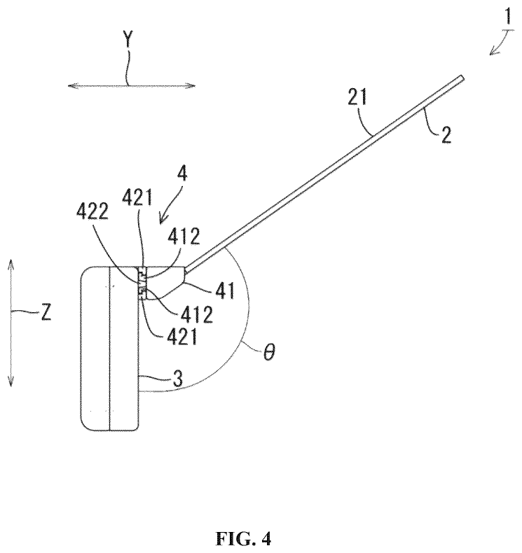

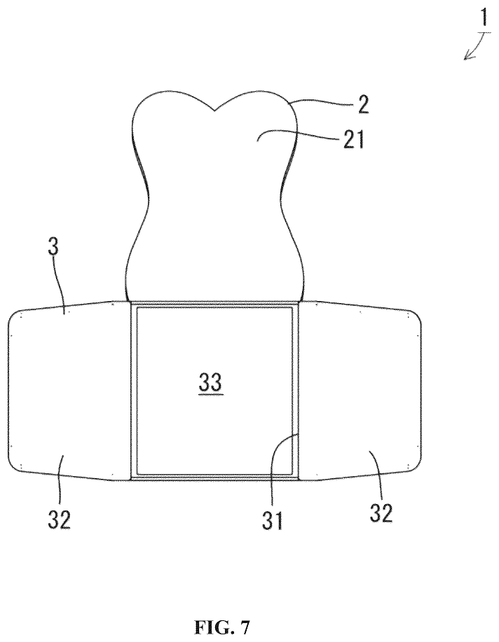

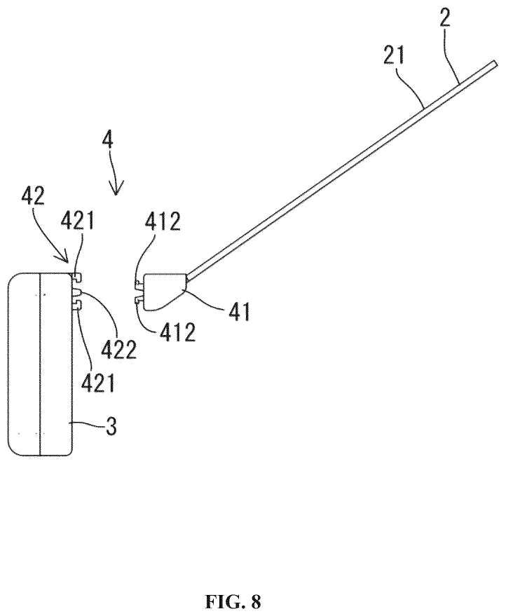

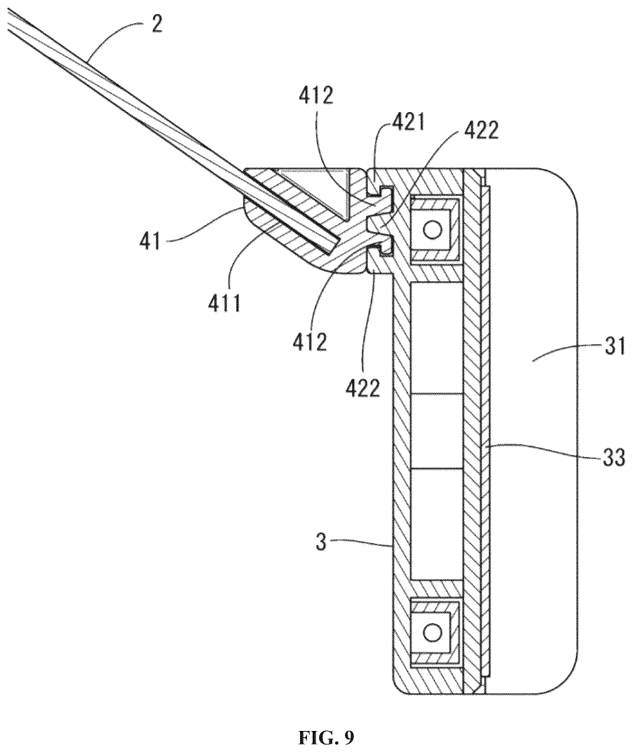

FIGS. 1A-10 show the first embodiment of the oral imaging aid of the present disclosure. As shown in FIGS. 1A, 1B, 2, 3, and 4, in this embodiment, the oral imaging aid 1 includes an insertion plate 2, a holder 3 that holds the mobile terminal 100, and a detachable attachment mechanism 4. For example, the detachable attachment mechanism may include a slidably detachable attachment mechanism or a magnetically detachable attachment mechanism.

When the mobile terminal 100 is held by the holder 3, the direction along the width of the mobile terminal 100 is defined as the horizontal direction X, the direction along the thickness of the mobile terminal 100 as the longitudinal direction Y, and the direction along the height of the mobile terminal 100 as the vertical direction Z.

The insertion plate 2 is an oral reflection means for imaging, which is inserted into the mouth to reflect the inside of the mouth, thereby enabling it to be imaged by the camera of the mobile terminal.

As shown in FIGS. 1A, 1B, 2, 3, 4, and 5, in this embodiment, the insertion plate 2 is formed from a metal plate such as stainless steel, is relatively thin, elongated on one side, and consists of a plate body with a predetermined area.

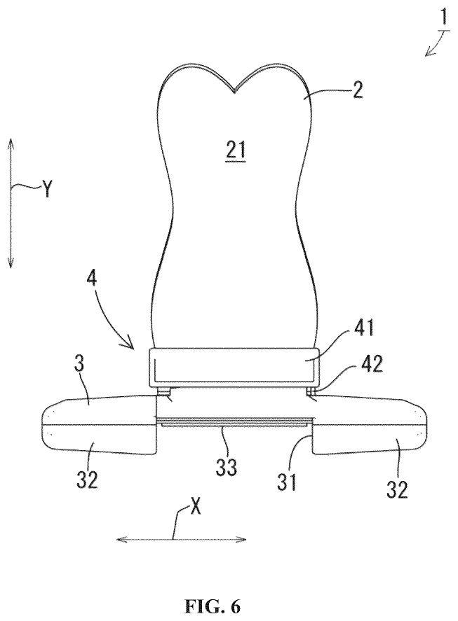

As also shown in FIG. 6, the insertion plate 2 has at least one side with a mirror finish to form a reflective surface 21. When the insertion plate 2 is inserted into patient's mouth, the inside of the mouth is reflected on this reflective surface 21. The reflective surface 21 of the insertion plate 2 is treated with an anti-fogging process, for example, by coating with a resin film or applying a clear anti-fogging agent.

The insertion plate 2 has a concave central part at its tip, and its sides are R-shaped, to make it a structure that is less likely to cause injury when inserted into the mouth. Furthermore, at an intermediate position along its longitudinal direction, the insertion plate 2 has a constricted part formed.

The size and shape of the insertion plate 2 may be arbitrary. For example, the insertion plate 2 may be categorized for a child, adult, male, female, elderly person, etc.

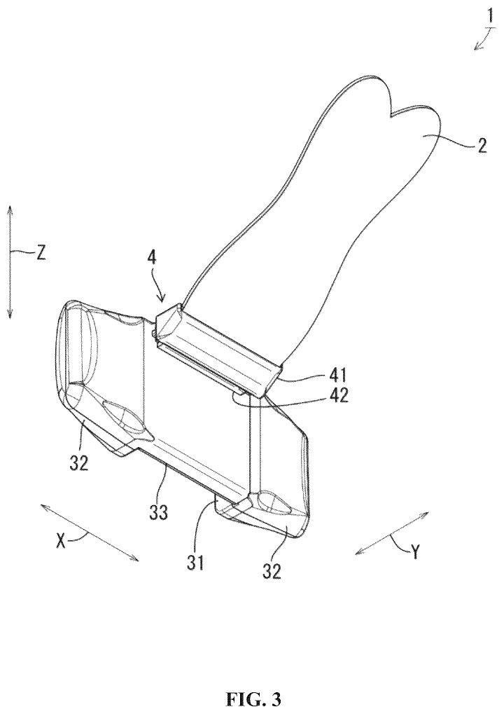

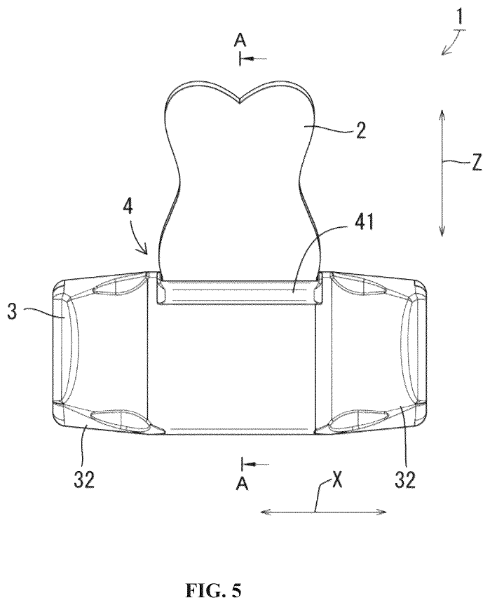

As also shown in FIGS. 3 and 5, the base end of the insertion plate 2 is detachably mounted on a clip 41, which will be described later.

The holder 3 is a mobile terminal fixing means that detachably secures a mobile terminal 100 having an imaging means such as a camera 101 or camera application. The holder 3 is mounted in such a way that the insertion plate 2 is inclined at a predetermined angle towards the front side through the detachable attachment mechanism 4.

The holder 3 mounts the mobile terminal 100 so that its camera 101 is directed towards the reflective surface 21 of the insertion plate 2, holding it at a predetermined angle θ between the mobile terminal 100 and the reflective surface 21 of the insertion plate 2 so that the reflective surface 21, which reflects the inside of the mouth, can be accurately imaged. In this embodiment, the insertion plate 2 is attached at an angle θ to the front surface of the holder 3.

The angle θ between the insertion plate 2 and the mobile terminal 100 may be set to more than 180 degrees and less than 270 degrees, for example. More preferably, the angle θ may be set within the range of about 210 to 240 degrees.

As shown in FIGS. 1A, 1B, 2, 3, and 10, in this embodiment, the holder 3 is formed from a hard synthetic resin such as a plastic and has a flat shape with a thickness in the longitudinal direction Y, consisting of a horizontally elongated octagonal solid. The shape of the holder 3 may be arbitrary, for example, rectangular, polygonal, circular, elliptical, etc.

The holder 3 has a mounting part 31 for the mobile terminal 100 formed at a rectangular part position near the approximate center in the horizontal direction X.

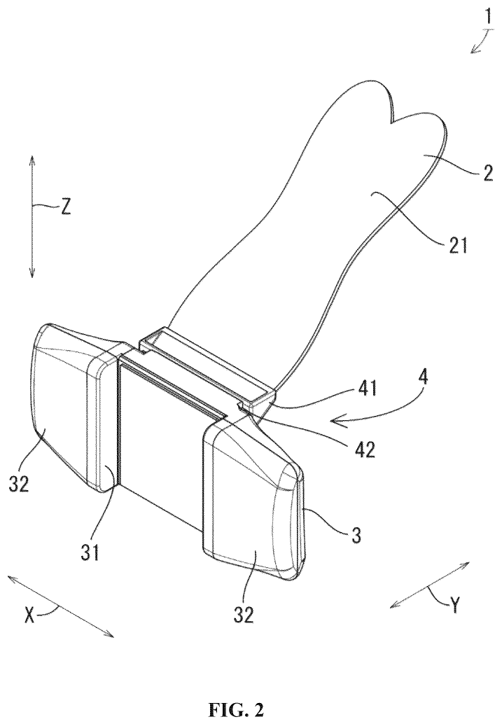

As shown in FIGS. 2, 6, and 7, the mounting part 31 is provided with a concave part formed by notching the rear side opposite to the side where the insertion plate 2 of the holder 3 is attached across the width of a mobile terminal such as a smartphone. The mobile terminal 100 is fitted into this mounting part 31 and fixed in place. The mounting part 31 has a holding surface 33 on the concave side. The holding surface 33 maintains the position of the mobile terminal 101 with adhesive or suction force.

The mounting part 31 allows for the adjustment of the position to mount the mobile terminal 100 along the vertical direction Z. In other words, the mounting part 31 is designed to allow for the position of the camera 101 of the mobile terminal 100 to be changed. This enables appropriate adjustments to be made according to the camera position of a mobile terminal such as a smartphone, which varies by model, to allow for accurate imaging of the inside of the mouth.

Additionally, the mounting part 31 may utilize any fixing means such as a hook, belt, magnet, or adhesive part as appropriate.

On both the left and the right sides of the holder 3, there are handle parts 32 that protrude in the horizontal direction X and are integrally formed so that the user can hold them with their hands. The handle parts 32 are formed in such a way that they gradually become narrower in both the vertical direction Z and the thickness direction Y from the central part of the holder 3 towards the outer sides, and the corner parts are rounded.

By providing handle parts 32 on the holder 3, the user can hold it stably with their hands and perform the imaging operation to allow for smooth and accurate adjustment of the insertion plate and the imaging position. The handle parts 32 also help to prevent camera from shaking or dropping during imaging.

Furthermore, the handle parts 32 of the holder 3 are not limited to the above-mentioned structure. For example, the handle parts 32 may have recesses that fit the shape of fingers or hands, be processed with rubber or unevenness to prevent slipping, be shaped to fit the fingers, or have any other structure that is easy to hold. Additionally, the handle part may be formed on only one side, either the left or right side of the holder.

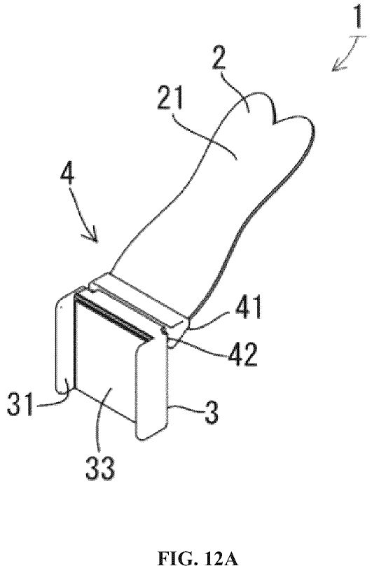

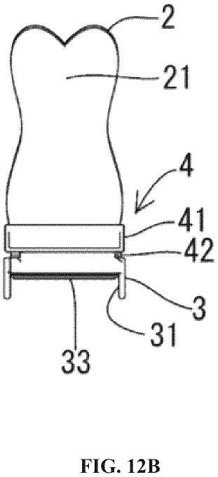

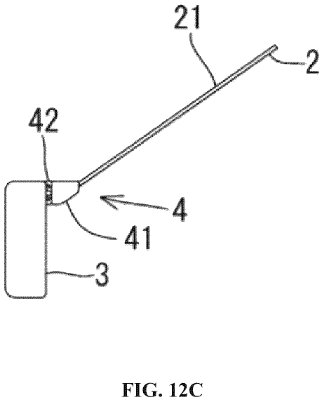

As shown in FIGS. 12A-12C, the holder 3 may have a simple structure without handle parts on both sides. In the configuration of the holder 3 without handle parts as shown in FIGS. 12A-12C, the user will hold it with one hand, which may be less stable during use compared to the above-mentioned embodiment. However, since the structure is simplified and compact, it can be manufactured at a low cost and is advantageous for carrying and storage.

The detachable attachment mechanism 4 is a mounting means that allows the insertion plate 2 to be detachably attached to the holder 3 at a predetermined appropriate angle. For example, the detachable attachment mechanism may include a slidably detachable attachment mechanism or a magnetically detachable attachment mechanism.

As shown in FIGS. 1A, 1B, 8, and 9, in this embodiment, the detachable attachment mechanism 4 includes, for example, a clip 41 for mounting the insertion plate 2 and a detachable guide 42 provided on the holder 3 side that can be detachably engaged with the clip 41. In this embodiment, the detachable attachment mechanism 4 is described as a slidably detachable attachment mechanism. The detachable attachment mechanism 4, which is a slidably detachable attachment mechanism, serves as a position adjusting means that allows the insertion plate 2 to be slidably moved relative to the holder 3 to adjust the mounting position. Even if the detachable attachment mechanism 4 is a magnetically detachable attachment mechanism, it detachably attaches the insertion plate 2 to the holder 3 at a predetermined angle. Furthermore, the detachable attachment mechanism 4 may be any configuration that allows the insertion plate 2 to be detachably attached to the holder 3 at a predetermined angle, such as a configuration that allows for detachable attachment in a mating manner or in a clamping manner, or any other suitable configuration.

The clip 41 is, for example, formed from a hard synthetic resin such as the same plastic forming the holder 3. The clip 41 has an insertion hole 411 in which the base end of the insertion plate 2 is inserted in a mating manner. The insertion hole 411 is recessed in an inclined direction relative to the holder 3 to set a predetermined angle θ. The upper side of the clip 41 has a hole formed.

On the rear side of the clip 41, for example, there is a protruding engaging member 412 that is formed in an approximately L-shaped cross-section and extends linearly in the lateral direction. Two engaging members 412 are provided parallel to each other facing in opposite directions in an approximate L-shape.

The detachable guide 42 has, for example, multiple rail parts 421 and 422 that protrude from the holder 3 and consists of that are provided to slidably detachably engage with the engaging members 412 of the clip 41. The engaging members 412 of the clip 41 are inserted into the gap between the rail parts 421 and 422, and the clip 41 with the insertion plate 2 mounted slides linearly in the lateral direction X along the rail parts (grooves) while maintaining the predetermined angle θ.

The detachable guide 42 is, for example, protruding from the holder 3 in an approximate L-shape and formed longitudinally in the horizontal direction X. The rail part 421 is engaged in a mating manner with the engaging member 412 of the clip 41. Additionally, the detachable guide 42 has an intermediate rail part 422 positioned between the opposing engaging members 412 of the clip 41. At both the left and the right ends of the rail part 421, guide tapers 423 are formed to smoothly insert the engaging members 412 of the clip 41.

The structure of the detachable attachment mechanism 4 is not limited to that described above. For example, the detachable attachment mechanism 4 may be configured to detachably engage the base end of the insertion plate directly with the holder 3 without using a clip. Furthermore, the detachable attachment mechanism 4 may also be configured to allow sliding movement not only in the horizontal direction X but also in the vertical direction Z.

Next, an example of how to use the oral imaging aid 1 according to this embodiment will be explained below. For example, the insertion plate 2, the clip 41, and the holder 3 may be separated from each other and sold or lent out to a user or a patient, etc., as a set stored in a storage box, etc. The mobile terminal 100 may be one owned by the patient or the patient's family, etc.

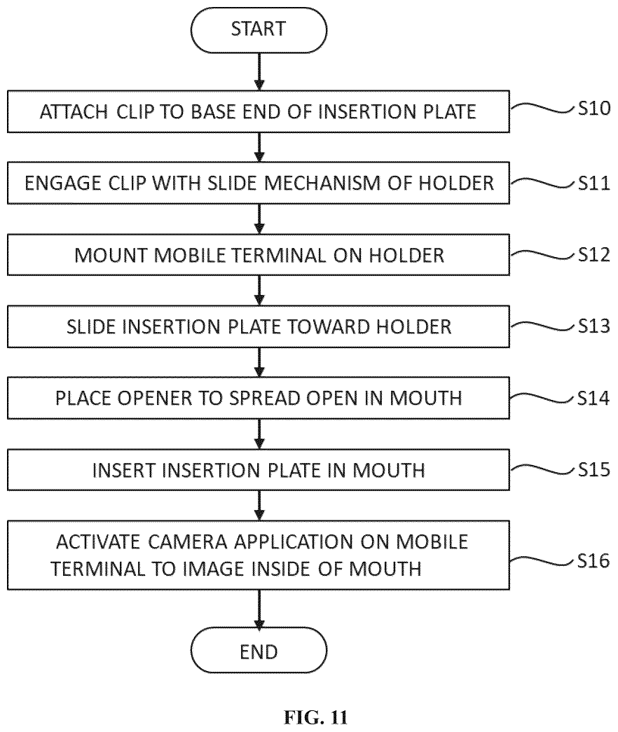

To image the inside of the mouth, each component is taken out of the storage box. At the step S10 of the flowchart shown in FIG. 11, the clip 41 is attached to the base end of the insertion plate 2.

Next, at the step S11, the engaging member 412 of the clip 41 is engaged with the detachable guide 42 of the holder 3.

In the following step S12, a mobile terminal 100 such as a smartphone is mounted on the mounting part 31 of the holder 3. At this time, if necessary, the mobile terminal 100 is slid in the vertical direction Z to adjust so that the camera of the mobile terminal 100 can appropriately image the reflective surface of the insertion plate 100.

Furthermore, at the step $13, if necessary, the insertion plate 2 and the clip 41 are slid in the horizontal direction X relative to the holder 3 through the detachable attachment mechanism 4 to adjust the position so that the camera of the mobile terminal 100 can appropriately image the reflective surface of the insertion plate 100.

At the step S14, an opener (not shown) is placed in the patient's mouth to spread it open.

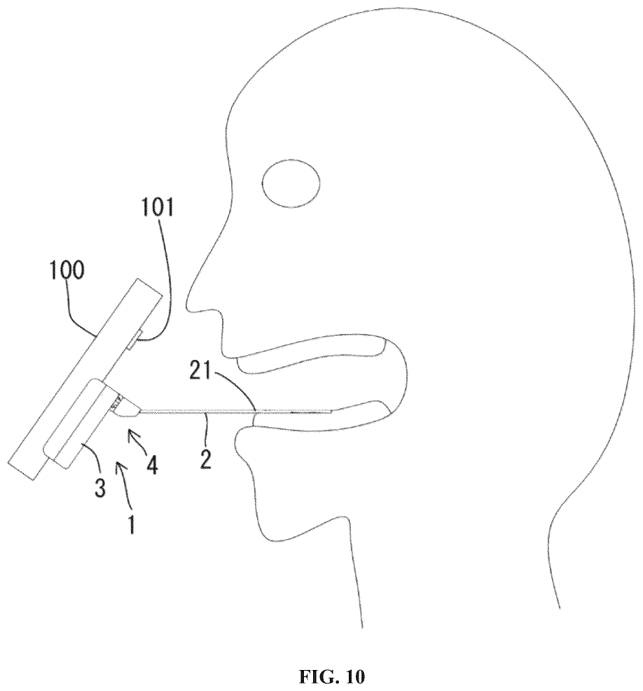

In the following step S15, as shown in FIG. 10, the patient her or himself holds and operates the holder 3 to align and insert the tip of the insertion plate 2 in the mouth.

Next, at the step S16, the camera application on the mobile terminal 100 is activated to image the inside of the mouth. First, with the reflective surface 21 of the insertion plate 2 facing upward, the insertion plate 2 is inserted into the mouth, pressed against the lower jaw, and stabilized, and then the condition of the upper jaw is imaged.

When the patient is imaging her or himself, for example, she or he may directly check the image displayed on the screen of the mobile terminal 100, or reflect the screen of the mobile terminal 100 on a mirror above a washbasin, etc., and take one or more images while confirming the image by looking at the screen.

Furthermore, by flipping the entire oral imaging aid 1 along with the mobile terminal 100 upside down, inserting the insertion plate 2 into the mouth with the reflective surface 21 of the insertion plate 2 facing downward, pressing the insertion plate 2 against the upper jaw, and stabilized, the same operations are performed to image the condition of the lower jaw.

In this way, even a patient her or himself can easily, smoothly, and accurately image the inside of the mouth. Even the patient's family member can also take the image more easily and accurately. Once imaging is completed (END), the images are sent to a hospital, doctor, etc., and oral examination is conducted online.

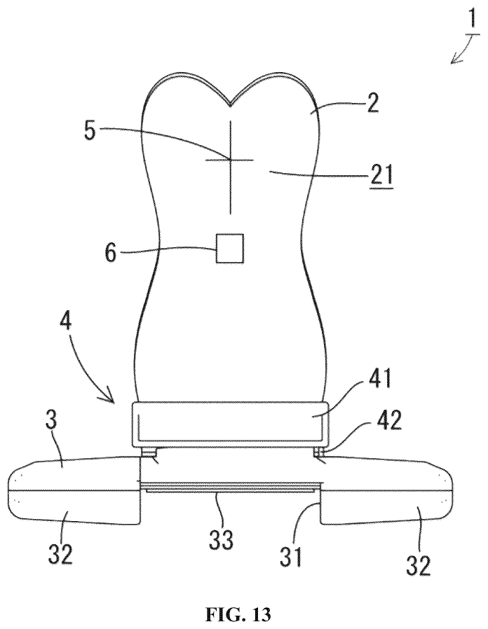

Additionally, FIG. 13 shows another embodiment of the insertion plate 2 of the oral imaging aid 1. As shown in FIG. 13, an alignment marker 5 for alignment in the mouth may be formed on the insertion plate 2. For example, when the teeth are imaged as the main subject, the marker 5 should be positioned or displayed in such a way that the marker 5 does not overlap with the subject.

Furthermore, the insertion plate 2 may be provided with a light source 6 for illuminating the inside of the mouth.

The oral imaging aid of the present disclosure described above is not limited to the configuration of the embodiments described above, and any modifications can be made without departing from the essence of the present disclosure as described in the claims.

### Industrial Applicability

The oral imaging aid of the present disclosure can be used to easily and accurately image the inside of the mouth at home and to transmit the image to a hospital or doctor for online remote medical examination, etc.

What is claimed is:

1. An oral imaging aid comprising:
an insertion plate that is inserted into a mouth, the insertion part having a reflective surface reflecting and displaying the inside of the mouth;
a holder that detachably mounts and holds a mobile terminal having an imaging means; and
a detachable attachment mechanism that detachably attaches the insertion plate to the holder at a predetermined angle.

2. The oral imaging aid according to claim 1, wherein the detachable attachment mechanism includes:
a clip mounted on the base end part of the insertion plate; and
a detachable attachment guide provided on the holder to detachably engage with the clip.

3. The oral imaging aid according to claim 1 or 2, wherein the holder is provided to allow the mounting position of the mobile terminal to be changed.

4. The oral imaging aid according to any one of claims 1 to 3, wherein a handle part is formed on either or both of the left and the right sides of the holder.

5. The oral imaging aid according to any one of claims 1 to 4, wherein the reflective surface of the insertion plate is treated with an anti-fogging process.

6. The oral imaging aid according to any one of claims 1 to 5, wherein the insertion plate is formed with an alignment marker for alignment in the mouth.

7. The oral imaging aid according to any one of claims 1 to 6, wherein the insertion plate is equipped with a light source for illuminating the inside of the mouth.
